(19) United States
(12) Patent Application Publication
(10) Pub. No.: US 2025/0082925 A1
(43) Pub. Date: Mar. 13, 2025

(54) SUBCUTANEOUS TRANSCRANIAL CORTICAL ELECTROTHERAPY STIMULATION METHOD AND DEVICE

(71) Applicant: Epic Neuro, Inc., Fountain Valley, CA (US)

(72) Inventors: James William PHILLIPS, Fountain Valley, CA (US); Robert M. ABRAMS, Los Gatos, CA (US)

(21) Appl. No.: 18/566,284

(22) PCT Filed: Jun. 1, 2022
(86) PCT No.: PCT/US2022/031741

§ 371 Date: Dec. 1, 2023

Related U.S. Application Data
(60) Provisional application No. 63/195,624, filed on Jun. 1, 2021.

(51) Int. Cl. A61N 1/05; A61N 1/36

(52) U.S. Cl. CPC A61N 1/0539; A61N 1/36139

(57) ABSTRACT

Systems and methods are described, which provides electrical stimulation to a person, where a current flow path is between a first electrode disposed below a scalp of the subject and outside a cranial cavity of the subject to a second electrode disposed below the scalp and outside the cranial cavity, and further comprises directing the current flow path through the subject's skull and into the brain tissue.

### PRIORITY CLAIM

This patent application claims priority to U.S. provisional patent application No. 63/195,624, titled “SUBCUTANEOUS TRANSCRANIAL CORTICAL ELECTROTHERAPY STIMULATION METHOD AND DEVICE” and filed on Jun. 1, 2021, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Electric brain stimulation has been shown to be a potentially effective treatment for a number of brain disorders, including epilepsy, migraine, fibromyalgia, major depression, stroke rehabilitation, and Parkinson's disease. External stimulation tends to be unfocused, and direct cortical stimulation is often highly invasive, involving a craniotomy or drill holes in the skull in order to target a specific cortical location. It would be beneficial to find a brain stimulation solution that will provide targeted cortical stimulation without requiring a surgical procedure which penetrates the skull.

Electric brain stimulation may be accomplished by several means. Repetitive Transcranial Magnetic Stimulation (rTMS) is a noninvasive technique that uses a coil to deliver a series of high energy magnetic pulses to the brain, thereby inducing current to flow in the cortex underneath the coil. rTMS has been shown to be effective in the treatment of major depression, and other mental disorders. However, it is not easily directed to a particular location, and involves a large, expensive device to generate the high current pulse to the coil. rTMS is not portable and requires a treatment administrator to deliver therapy to the patient.

Transcranial Direct Current Stimulation ((DCS) uses electrodes on the outside of the head to deliver small amounts of current to the brain. tDCS was originally used for stroke recovery, and it has shown promise in the treatment of some mental disorders and for cognitive improvement. Electrodes are placed on skin surfaces on the outside of the subject's head near the region of interest for stimulation. The vast majority of current is shunted between the electrodes since the skull is a very effective electrical insulator. However, a portion of the current does result in intracerebral current flow, which may increase or decrease neuronal excitability and alter brain function. The exact method of action is unclear. tDCS current strength is limited due to the excitability of nerves in the scalp, which can cause discomfort to the patient if the current is set too high.

Vagus nerve stimulation involves electrically stimulating the vagus nerve in the neck of the patient. This can be done either using electrodes on the skin, which may involve painful sensation of the patient, or surgically implanting electrodes near the vagus nerve, generally with a power source implanted elsewhere in the body. This involves a significant surgical procedure and has shown efficacy in treatment of epilepsy and depression.

Deep brain stimulation (DBS) uses electrodes implanted and placed bilaterally into the basal ganglia, cerebellum, anterior principal nucleus, the centromedian nucleus, caudate nucleus, thalmic, or subthalmic region. Stimulation may also be delivered subcortically. Stimulus trains are delivered for treatment of a number of disorders, including epilepsy, Parkinson's disease, and major depression. DBS is generally a very invasive procedure, requiring a long lead that penetrates the skull with multiple electrodes near the tip. The procedure is considered major surgery and is not generally used unless other methods have been exhausted.

Direct cortical stimulation (DCS) is similar to DBS, except that the lead lies on the surface of the cortex, either subdural or epidural. The electrodes are secured in place using sutures. This technique often involves removing a portion of the skull to gain access to the cortical surface, and possibly to make room for the power source. DCS has been shown to have efficacy in treatment of epilepsy and neuropathic pain. (Shanechi et al., 2013) introduced a Brain-Machine Interface that uses EEG to automatically titrate drugs during a medically induced coma. (Liu et al., 2006) automatically adjusted anesthetic during a surgical procedure using Bispectral index (BIS) calculated from the EEG. The company Aspect Medical, Inc. was created to develop a device for this application. Also, Drager Medical, Inc. developed the Zeus for closed-circuit anesthesia ventilation. (Doufas et al., 2003) used an automatic response test for optimizing propofol administration during conscious sedation. Phillips (U.S. Pat. Nos. 9,872,996, 10,780,286) uses a subcutaneous pulse generator and a conductive path through the skull at multiple locations to create a current-loop. The Phillips method and device still involves at least two drill-holes in the skull.

Clearly, it would be beneficial to have a device which allowed for relatively focal minimally invasive cortical stimulation without requiring major surgery.

### SUMMARY OF THE DISCLOSURE

A device for electrical stimulation of a subject's brain, the device comprising a first electrode adapted to be implanted under a scalp of the subject and outside the subject's cranial cavity, a second electrode adapted to be implanted under the subject's scalp and outside the subject's cranial cavity, insulating material surrounding a portion of the first electrode, the insulating material having an impedance higher than an impedance of a human skull, and a current generator adapted to generate an electric current between the first electrode and the second electrode.

In some embodiments, the device includes an insulating material surrounding a portion of the second electrode, the insulating material having an impedance higher than the impedance of a human skull.

In one example, the first electrode and the insulating material are disposed on a first side of the current generator.

In another example, the second electrode is disposed on the first side of the current generator.

In some embodiments, the second electrode is disposed on a second side of the current generator.

In some embodiments, the insulating material extends a uniform distance from a circumference of the first electrode.

In one embodiment, the insulating material extends in non-uniform distances from a circumference of the first electrode.

In some embodiments, the electrode comprises a screw adapted to be screwed into the subject's skull.

A method for providing electrical stimulation to brain tissue of a subject is provided. the method comprising generating a current flow path between a first electrode disposed below a scalp of the subject and outside a cranial cavity of the subject to a second electrode disposed below the scalp and outside the cranial cavity, and directing the current flow path through the subject's skull and into the brain tissue.

In some embodiments, the directing step comprises resisting current flow between the first electrode and the second electrode outside of the skull with insulating material having an impedance higher than an impedance of the skull.

In some embodiments, the insulating material surrounds a portion of the first electrode.

In another embodiment, the insulating material surrounds a portion of the second electrode.

In some embodiments, the method further comprises placing the insulating material against an outside surface of the skull between the first electrode and the second electrode.

In one example, the generating step comprises generating current with a current generator disposed between the scalp and the skull.

In another embodiment, the method further comprises screwing the first electrode into the skull.

In some embodiments, the method includes creating an opening through the skull to the cranial cavity adjacent the first electrode or the second electrode.

A method for obtaining an EEG of a subject's brain is provided, the method comprising sensing an elecroencephelography signal from a first electrode disposed below a scalp of the subject and outside a cranial cavity of the subject and a second electrode disposed below the scalp and outside the cranial cavity, and recording the electroencephelography signal.

In some embodiments, at least a portion of the first electrode and/or the second electrode is surrounded by an insulating material.

### BRIEF DESCRIPTION OF THE DRAWINGS

FIG. 1 is an example of two electrodes positioned near the skull and under the scalp.

FIG. 2 another example similar to that of FIG. 1 except insulation extends over to the side of the electrodes.

FIG. 3 is an example where a cathode is positioned near the skull and is covered and surrounded by an insulator.

FIG. 4 shows a brain stimulation device that comprises an implantable current generator.

FIG. 5 is a bottom view of the device of FIG. 4.

FIG. 6 is an example of a device where the current will flow from the central cathode to the ring-shaped anode.

FIG. 7 shows an example of an insulator which comprises a notch in order to increase current density in that region of the brain underneath the notch.

FIG. 8 is an example of a device where electrodes include a screw-shaped cathode twisted into the skull.

FIG. 9 shows a device with a current generator implanted beneath the scalp.

FIG. 10 is an example of a device with the central region between the electrodes clear of insulation without significantly reducing the percentage of current that enters the sub-cranial space.

FIG. 11 shows a device with a current generator configured to prevent current from flowing from the cathode to the anode through the current generator.

FIG. 12 is a cross-sectional view of a device with a current generator configured to prevent current from flowing from the cathode to the anode through the current generator.

FIG. 13 shows a cross-sectional view of two implanted brain stimulation devices.

FIG. 14 is a device having an insulator with a higher impedance than the bone in the skull.

FIG. 15 shows an example device in which both electrodes are insulated from each other and from the scalp tissue, exposed only on the side that is near the skull.

FIG. 16 shows a bottom view of the device of FIG. 15.

FIG. 17 is a cross-sectional view of a brain stimulation device.

FIG. 18 is an embodiment of a devie with a current generator external to the body.

FIGS. 19a-19c are examples of a brain stimulation model.

FIGS. 20a-20c are maps of the current density of the three models shown in FIGS. 19a-19c.

FIGS. 21a-21c are maps of the current density for the three models shown in FIGS. 19a-19c.

### DETAILED DESCRIPTION

It is possible to implement a fully subcutaneous brain stimulator which comprises two subcutaneous electrodes resting under the scalp of the person and on or near the skull. In such a system, the vast majority of the current between the two electrodes would shunt through the scalp, resulting in very little current actually flowing through the cortical tissue. The reason for this is because the skull is highly resistive to electrical current flow, 8-10 times less conductive than cerebrospinal fluid (CSF) or scalp tissue.

The present invention provides an electrically insulating material (such as, e.g., bone void filler) that has a far higher impedance than the skull's impedance) around and/or between the electrodes to direct the current to flow into and through the skull to the brain tissue in the sub-cranial space (cranial cavity) beneath the skull. In one embodiment, the insulator is malleable and acts like spackle or putty, adhering and hardening to the skull surface to hold the electrodes in place while preventing the shunting of electric current flowing between the electrodes from passing through the scalp, thereby directing the current to flow through the skull into the subject's brain. The malleable insulator may be applied during the implantation procedure. For example, a small flat blade may be used to apply the malleable insulator to the device around the electrodes before the device is inserted and pressed to the skull, or the blade may be used to apply insulator once the electrodes are in place. Alternately, the malleable insulator may be pre-applied to the device around the electrodes, before the procedure. In an alternative embodiment, the insulator may be a non-malleable insulator, such as a soft silicon material, which conforms to the skull surface. The non-malleable insulator may adhere to the skull surface on its own, or the non-malleable insulator may use an adhesive, such as glue, to adhere it to the skull surface, or it may use a mechanical means, such as screws, to adhere it to the skull surface. The skull is on average approximately 5 mm thick. Therefore, if, for example, the insulation is at least 10 mm wide, then the lowest impedance path for the current to travel from one electrode to the other is to proceed through the skull, entering the epidural space, with current flow in and around the cortex, then to flow back through the skull to the return electrode.

Either one or both electrodes could be insulated in this manner. The percentage of current that flows into the sub-cranial space is dependent on the skull thickness and the distance the current must travel through the skull when traveling through the sub-cranial space compared to the distance the current must travel through the skull if the current shunts through the skull between the stimulation electrode and return electrode. If the distance through the skull the current must travel when traveling through the sub-cranial space is lower than the distance the current must travel through the skull when not going through the sub-cranial space, then a greater percentage of the electrical current will flow through the sub-cranial space, thereby affecting the cortex of the brain.

In the examples that follow, one electrode is generally designated as the cathode (+) and one electrode is designated as the anode (−). This is for convention, with arrows showing the direction of current flow. This should not be considered limiting, as the anode and cathode may be switched, while still retaining the relevance of the example.

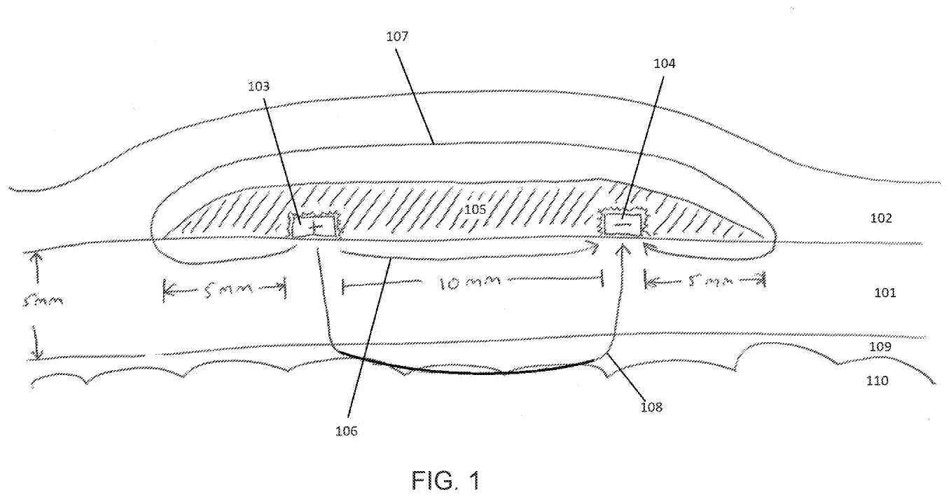

One example is shown in FIG. 1. In this example, two electrodes are positioned near to the skull (101) and under the scalp (102). One electrode (103) is the cathode and one electrode (104) is the anode. The electrodes are 10 mm apart. The electrodes are covered and surrounded by an insulating material (105), which extends over, and 5 mm on each side of, the electrodes. The insulating material has a higher impedance than the impedance of the bone in the skull. When an electric current is generated by a current generator (not shown) from the cathode (103) to the anode (104), the insulating material ensures that the current must travel through the skull for at least a portion of the path. The current flow follows three general paths. The current path (106) could flow entirely through bone of the skull (101), requiring the current to travel at least 10 mm through bone. The current could flow along a current path (107) through bone, through the scalp (102) covering the insulating material, and back through bone, requiring the current to travel at least 10 mm through bone and 20 mm through the scalp tissue. Lastly, the current could flow along a current path (108) into the sub-cranial space (109), through the brain (110), and back through bone. This would also require the current to travel at least 10 mm through bone. Therefore, a significant amount of the supplied current, though not necessarily the majority, will follow the sub-cranial path (107), thereby affecting the cortical tissue beneath the electrodes.

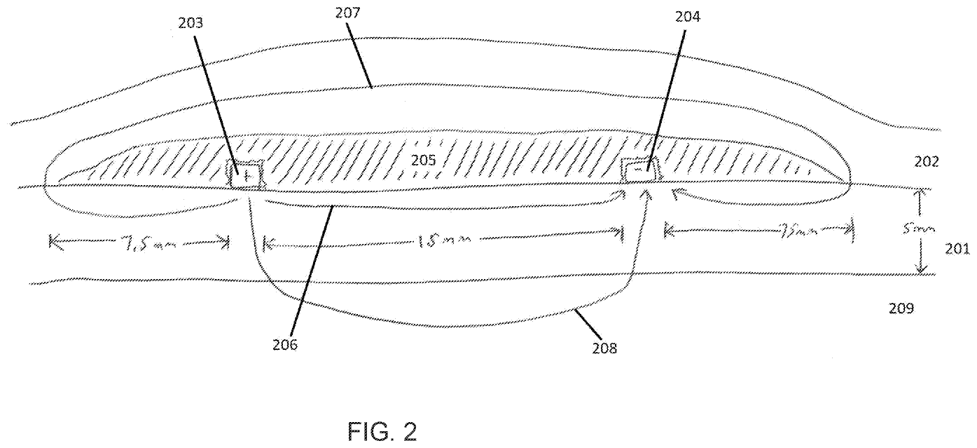

Another example is shown in FIG. 2. This is similar to the example from FIG. 1. except that the electrodes (203) and (204) are 15 mm apart, and the insulation (205) extends over and 7.5 mm to the side of the electrodes. Everything is subcutaneous, situated under the scalp (202). When an electric current is generated by a current generator (not shown), the current path (206) flows entirely through bone (201) and must travel at least 15 mm through bone. The current path (207) would flow through bone, under the scalp, and back through bone, requiring at least 15 mm through bone as well. However, the current path (208) flows through 5 mm of bone, through the sub-cranial space (209), then back through bone, requiring only a minimum of 10 mm through bone. In this case, a greater percentage of the current will flow through the sub-cranial space than in the FIG. 1 embodiment because the thickness of the skull remains 5 mm while the distance through bone required to avoid the sub-cranial space has increased. Following this logic, the wider the skull distance between the electrodes, the more current will be directed to the sub-cranial space, thereby affecting the cortical tissue to a greater degree.

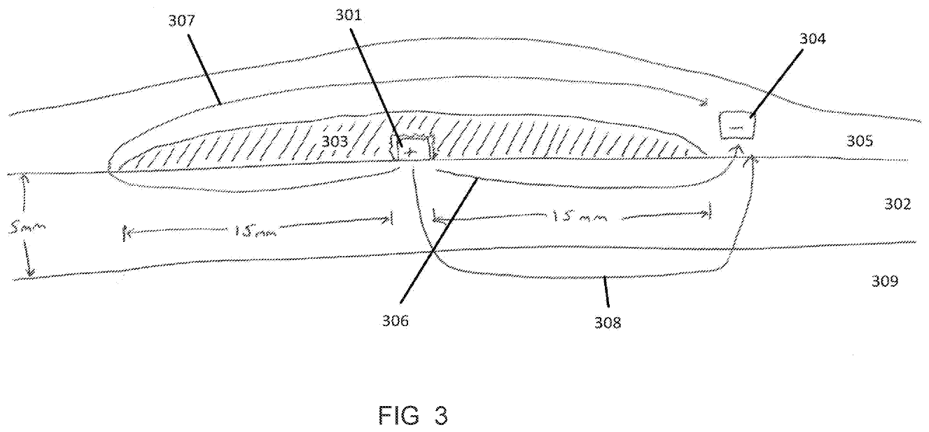

Another example configuration is shown in FIG. 3. In this example, the cathode (301) is positioned near the skull (302) and is covered and surrounded by an insulator (303). The anode (304) is positioned in the scalp (305). The anode may be next to the skull or above the skull. The skull is 5 mm thick, and the insulator extends 15 mm on all sides of the cathode. Similar to the previous two examples, when an electric current is generated by a current generator (not shown), the current is distributed among three current paths. In the first current path (306), the current travels from the cathode (301) entirely through the skull to the edge of the insulator (303), and then enters the tissue and proceeds to the anode (304). In the second current path (307), the current flows the opposite direction to the edge of the insulator, then enters the scalp and flows back over the insulator to the anode. In the third current path (308), the current moves through the skull and into the sub-cranial space (309), then back through the skull and into the tissue to the anode. As can be calculated, the first two current paths involve 15 mm through bone, whereas the third path involves 10 mm through bone. Therefore, due to the relatively high impedance of bone compared to the impedance of the insulating material, it can be expected that a significant portion of the current density will flow through the sub-cranial space, affecting the cortical tissue.

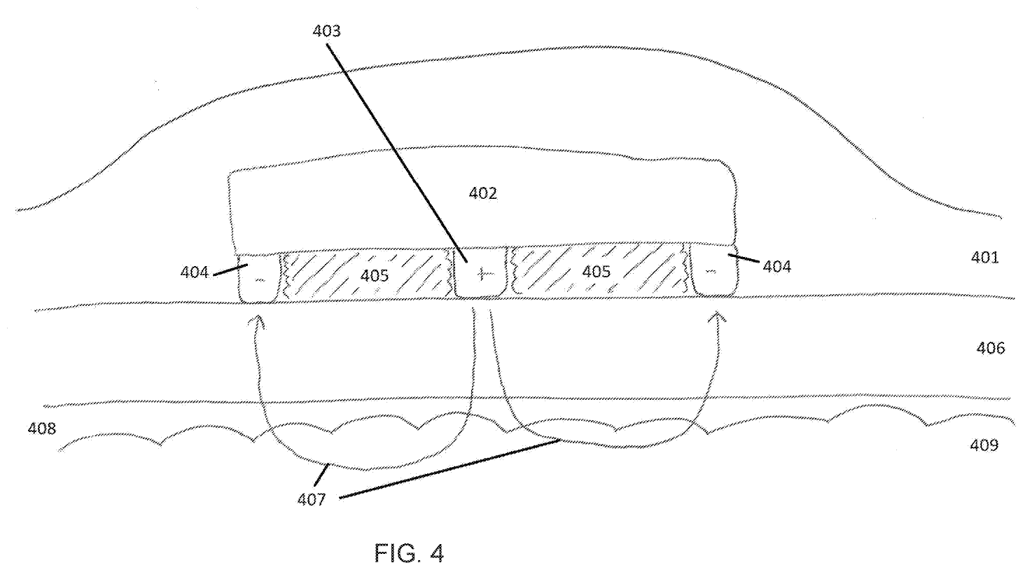

An example brain stimulation device is shown in FIG. 4. In this example, the device is implanted under the scalp (401) and comprises an implantable current generator (402). The device in this example is circular, and FIG. 4 shows a cross-section of the device. The device comprises a central cathode (403) and a ring-shaped anode (404) that is connected to the bottom edge of the implantable current generator. An insulator (405) is also ring-shaped, and fills the space between the central cathode (403) and the anode ring (404). The insulator has an impedance higher than the impedance of the bone in the skull (406). The current generator is configured to prevent current from flowing from the cathode to the anode through the current generator. The device is positioned so that the insulator is pressed near the skull (406), such that current cannot shunt from the cathode to the anode without proceeding through the skull. The current generator causes current to flow from the cathode to the anode. Although a portion of the current will shunt through the skull from the cathode to the anode, a significant current density will proceed along the current path (407) through the skull, then into the sub-cranial space (408) and the brain (409) before returning through the skull back to the anode.

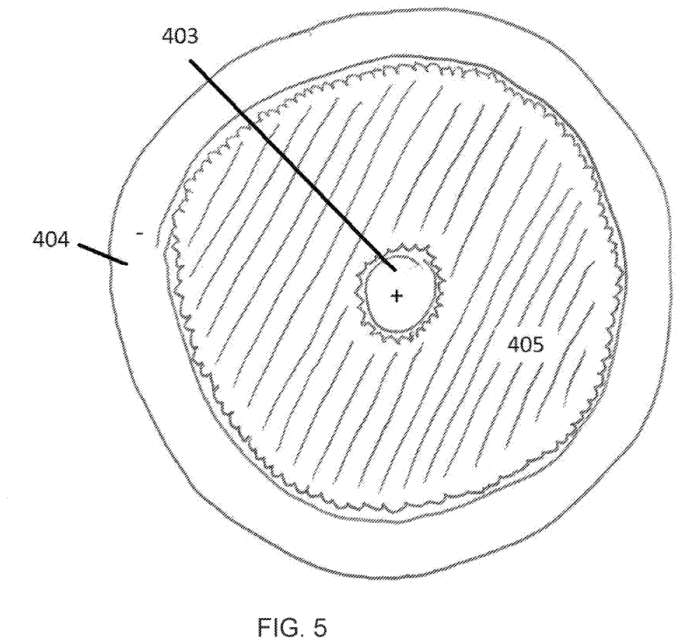

FIG. 5 shows a bottom view of the device in FIG. 4. The cathode (403) is shown in the center of the circular-shaped device. A ring-shaped anode (404) occupies the outer edge of the device. An insulator (405) fills the space between the cathode and the anode ring.

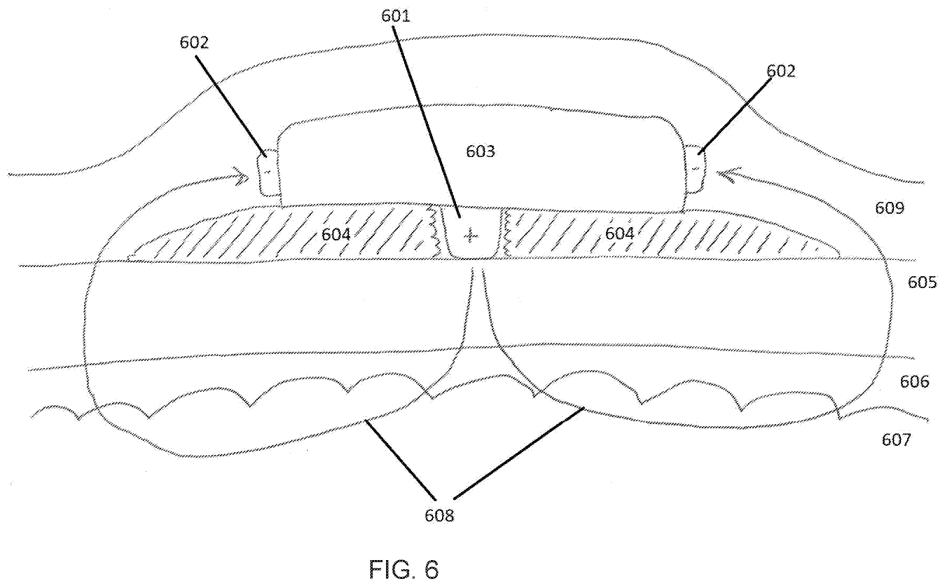

Another example of a brain stimulation device is shown in FIG. 6. This example is similar to the one in FIG. 4, in that figure shows a cross-section of the device, and the device comprises a central cathode (601) and a ring-shaped anode (602). In this example, the anode is on the side of the current generator (603). The current generator is configured to prevent current from flowing from the cathode to the anode through the current generator. The insulator (604) is near the skull (605), and extends out past the edge of the current generator. This extra width increases the minimum length of skull that current must cross in order to shunt from cathode to anode without entering the sub-cranial space (606). Once again, the insulator (604) has an impedance higher than the impedance of the bone in the skull (605). In this example, a significant percentage of the current will proceed along the current path (608) from the cathode, through the skull to the sub-cranial space and the brain (607), then back through the skull, through the scalp tissue (609) and to the anode.

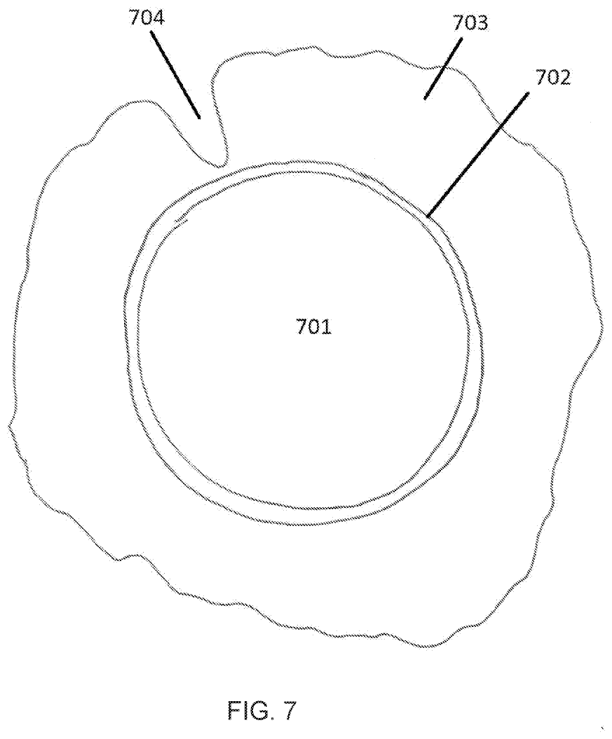

For the example in FIG. 6, the current will flow from the central cathode to the ring-shaped anode. The current density distribution will likely not be uniform around the ring. It will be affected by impedance changes in the skull, the surrounding tissue, or the CSF. For example, if the skull is thinner near one edge of the device, current density is likely to be higher in that region, due to the lower electrical impedance. It is possible to direct current flow somewhat, by varying the shape of the insulator. FIG. 7 shows an example of an insulator which comprises a notch, in order to increase current density in that region of the brain underneath the notch. As before, the insulator has an impedance higher than the impedance of the bone in the skull. The figure shows a top view of the device, in which a current generator (701) is surrounded by a ring anode (702). The cathode (not shown) is beneath the pulse generator and anode and is surrounded by the insulator (703), as in the embodiment of FIG. 6. The current generator is configured to prevent current from flowing from the cathode to the anode through the current generator. A notch (704) in the insulator increases the current density in the region of the brain underneath that notch. However, it may also increase the amount of current which is shunted straight through the skull, due to the reduced minimum distance in the skull that the current must travel to avoid going into the sub-cranial space.

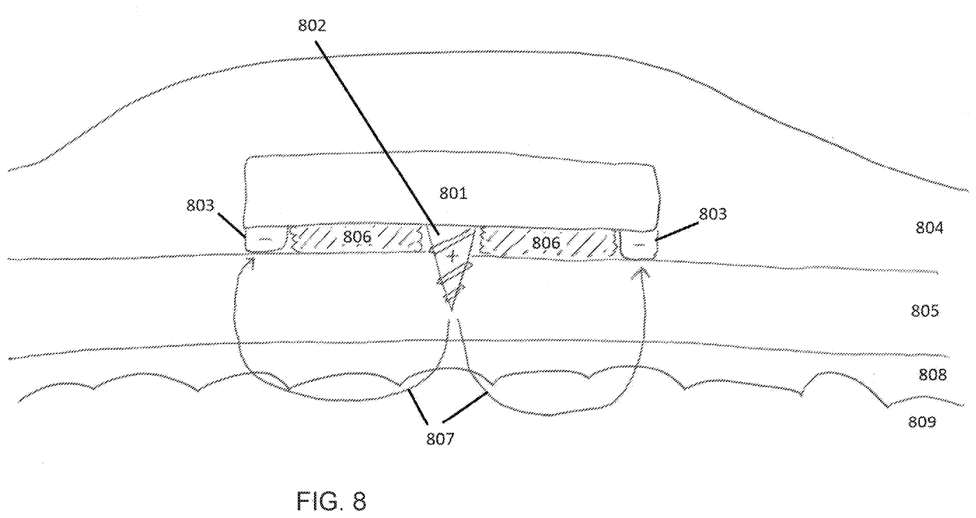

It is not necessary that the electrodes be completely outside the skull surface. It would be possible to penetrate a portion of the distance without involving the difficult procedure of creating a drill hole to the sub-cranial space. One example is shown in FIG. 8. A cross section of the device is shown of the device, which comprises a current generator (801), a central cathode screw (802) and a ring-shaped anode (803), and is implanted beneath the scalp (804). In this example, the screw-shaped cathode is twisted into the skull (805) to a depth dependent upon the length of the screw. In order to make insertion of the screw easier, a pilot hole may be drilled into the skull. Also, the top of the device may comprise a notch, such that a screwdriver could be used to facilitate twisting the device screw into the skull. An insulator (806) is positioned between the anode and cathode, and is pressed to the skull when the screw is twisted. Once again, the insulator (806) has an impedance higher than the impedance of the bone in the skull (805). The current generator is configured to prevent current from flowing from the cathode to the anode through the current generator. When current is generated, a significant portion of the current will follow along the current path (807), which proceeds from the screw-shaped cathode, through the skull, and into the sub-cranial space (808), through a portion of the brain (809), then back through the skull to the anode. The screw-shaped anode serves to hold the device in place, preventing the device from drifting or shifting across the skull surface. It also ensures that the insulator is held tight to the skull surface. In addition, the screw reduces the amount of skull the current must travel in order to enter the sub-cranial space, increasing the percentage of current affecting the brain. Care must be taken, however, to ensure the screw does not fully penetrate the skull, because the screw tip may puncture the dura and cause neural damage. To use the screw-shaped cathode, it is preferred to know the thickness of the skull into which the cathode must be twisted, so that the screw length or device location may be selected to ensure the screw length is less than the skull thickness.

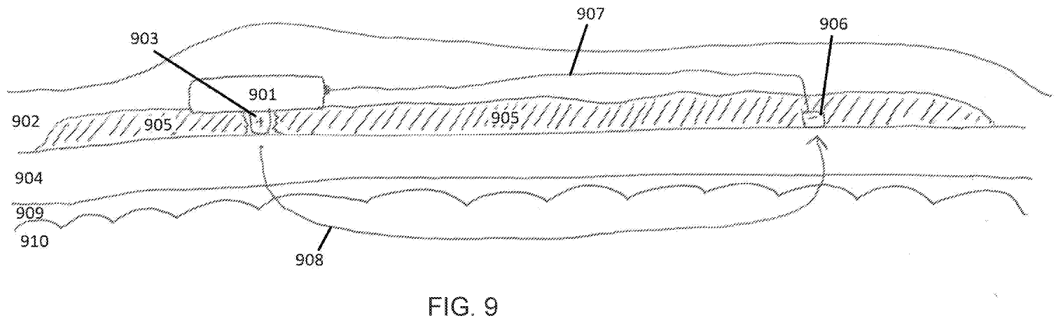

It is not necessary for both electrodes to be attached to the current generator, only that an electrical connection is made. One example is shown in FIG. 9, in which a current generator (901) is implanted beneath the scalp (902). A cathode (903) is positioned near the surface of the skull (904) and is surrounded by an insulator (905). Once again, the insulator (905) has an impedance higher than the impedance of the bone in the skull (904). An anode (906) is positioned near the skull and at a distance from the current generator (which is configured to prevent current from flowing from the cathode to the anode through the current generator), and is also surrounded by the insulator, which prevents electric current to shunt between the electrodes without entering the skull. The anode is electrically connected to the current generator by an insulated wire (907). When current is generated, a significant amount will follow the current path (908) flowing from the cathode, through the skull and into the sub-cranial space (909) and into the brain (910), then back through the skull to the anode. One advantage to this configuration is that the electrodes can be separated by a significant distance, which increases the area of the brain affected by the electric current, and it increases the percentage of current which is forced to flow into the sub-cranial space as opposed to shunting through the skull. It is important in this example to have the insulator extend beyond the electrodes a distance, in order to minimize the current which shunts from the cathode through the skull into the scalp, then back into the skull and to the anode.

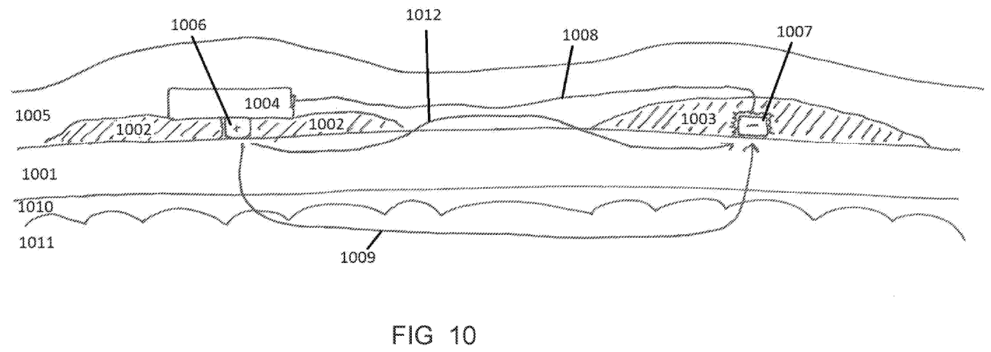

It is possible to leave the central region between the electrodes clear of insulation without significantly reducing the percentage of current that enters the sub-cranial space. An example of this is in FIG. 10, which is identical to FIG. 9, except that a portion of the skull (1001) is not covered by an insulator (1002, 1003). A current generator (1004) is implanted beneath the scalp (1005). A cathode (1006) is positioned such near the skull surface and is surrounded by an insulator (1002). An anode (1007) is positioned near the skull and at a distance from the current generator, and is also surrounded by a separate insulator (1003). Both insulators have impedances higher than the impedance of the bone in the skull, and the current generator is configured to prevent current from flowing from the cathode to the anode through the current generator. The anode is electrically connected to the current generator by an insulated wire (1008). When current is generated, a significant amount will follow the current path (1009) flowing from the cathode, through the skull and into the sub-cranial space (1010) and into the brain (1011), then back through the skull to the anode. An alternate current path (1012) also exists in this configuration where the current travels from the cathode, through a portion of the skull, into the scalp between the electrodes, then back through the skull and to the anode. Current flow in this alternate path may be minimized by increasing the coverage of the insulator (1002, 1003) between the electrodes.

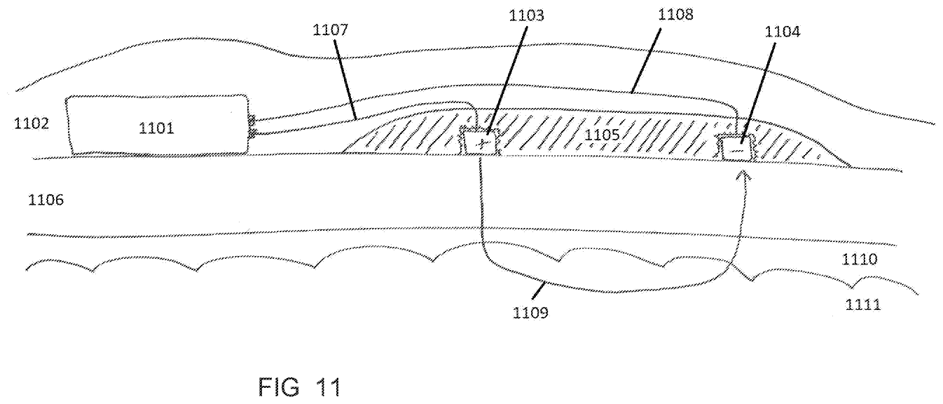

It is not necessary for the current generator to be connected to one or both of the electrodes. One example of this is shown in FIG. 11. In this, a current generator (1101) is implanted under the scalp (1102). Also implanted are two electrodes: the cathode (1103) and the anode (1104). The current generator is configured to prevent current from flowing from the cathode to the anode through the current generator. Both electrodes are covered by an insulator (1105) having an impedance higher than the impedance of the bone of the skull (1106), such that electric current must enter the skull (1106) to pass from the cathode to the anode. The cathode is connected to the current generator by an insulated wire (1107), and the anode is connected to the current generator by another insulated wire (1108). These wires may be held together as part of a lead, or they may be separate, as shown. When current is generated, a significant amount will follow the current path (1109) flowing from the cathode, through the skull and into the sub-cranial space (1110) and through the brain (1111), then back through the skull to the anode. In this example, the current generator is implanted beneath the scalp. However, it is also possible for the current generator to be implanted in another area. For example, the current generator may be implanted in the pectoral region, with a lead comprising the two wires (1107, 1108) tunneled under the skin, of the chest, shoulder, neck, and scalp, connecting finally to the two electrodes. One current generator may provide current to more than one pair of electrodes. For example. multiple electrodes may be implanted, and the current generator could supply current to various combinations of those electrodes, resulting in current flow that targets specific brain regions.

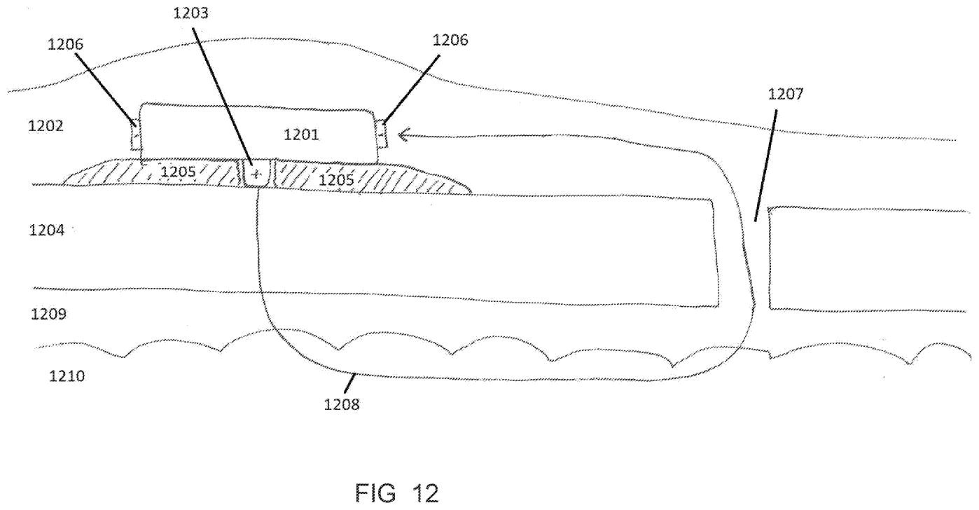

Although the device implantation procedure is simpler if the skull is not penetrated, it may be advantageous to drill a hole in the skull as part of the process. In this way, current may be better directed and controlled, overall impedance may be lowered, and a greater percentage of the generated current may enter the sub-cranial space to stimulate the brain. One example is shown in FIG. 12. This example shows a cross section of the device. In this example, a disk-shaped current generator (1201) is implanted beneath the scalp (1202). A cathode (1203) is attached beneath the current generator so that it rests near the skull (1204). The current generator is configured to prevent current from flowing from the cathode to the anode through the current generator. The cathode is surrounded by an insulator (1205). The insulator (1205) has an impedance higher than the impedance of the bone in the skull, which prevents current from flowing to the ring-shaped anode (1206) without entering the skull. A drill hole (1207) is positioned in a location that will allow current to flow through a target region of the brain. When current is generated, a significant amount will follow the path (1208) flowing from the cathode, through the skull and into the sub-cranial space (1209) and through the brain (1210), then through the drill hole, under the scalp and back to the ring-shaped anode. The drill hole may be left unoccupied, or it may contain a sleeve in order to prevent the bone from filling the drill hole naturally. Since the scalp comprises sensory nerves, the current flow through the scalp may be felt by the subject as a tingling or pinprick sensation, and the current may be reduced to account for comfort of the subject. This example shows a single drill hole. However, multiple drill-holes may be used to distribute current throughout different areas of the brain.

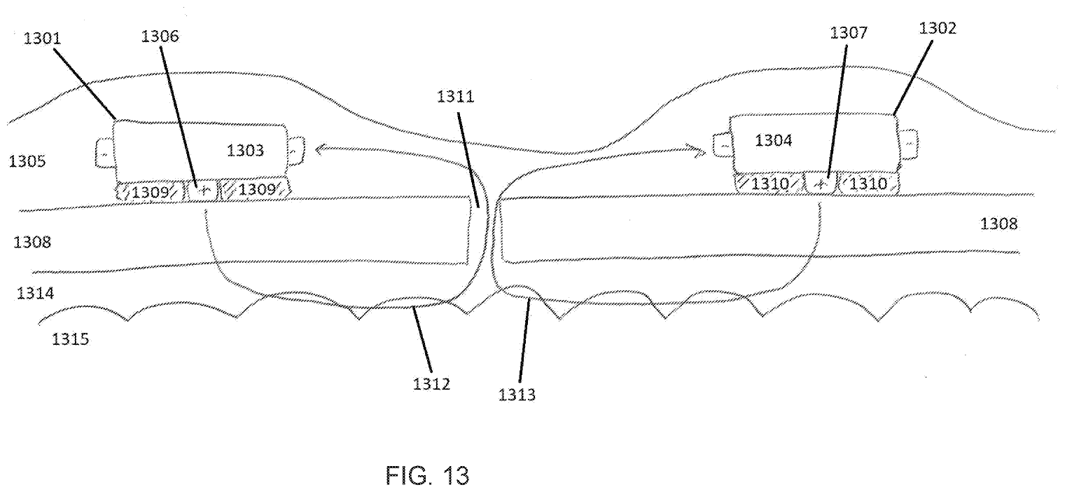

More than one device of the present invention may be implanted in a subject, which may allow for stimulation of a larger region or target certain locations in the brain. A drill hole may be used to further direct current flow, to lower the overall impedance. and to increase the percentage of current that flows into the subcranial space. One example is shown in FIG. 13, which shows a cross section of two implanted devices. Each device (1301, 1302) comprises a current generator (1303, 1304) which is implanted under the scalp (1305). A cathode (1306, 1307) is attached to the current generator and is positioned near the skull (1308). The current generators are each configured to prevent current from flowing from its cathode to its anode through the current generator. Insulators (1309, 1310) each having an impedance higher than the impedance of the bone in the skull are situated around the cathodes so that nearly all current from the cathodes is forced into the skull. A drill hole (1311) in the skull is positioned between the two devices. When current is generated, a significant amount will follow the current paths (1312, 1313) from the cathode, through the skull into the sub-cranial space (1314) and through the brain (1315), then through the drill hole into the scalp and back to the anode. More drill holes or more devices may be used to further direct electric current flow. In one possible application, devices could be implanted in the perilesional area of a stroke patient, with a drill hole positioned at the central point of the lesion itself. In this way, electric current may flow through the entire lesion as part of stroke rehabilitation.

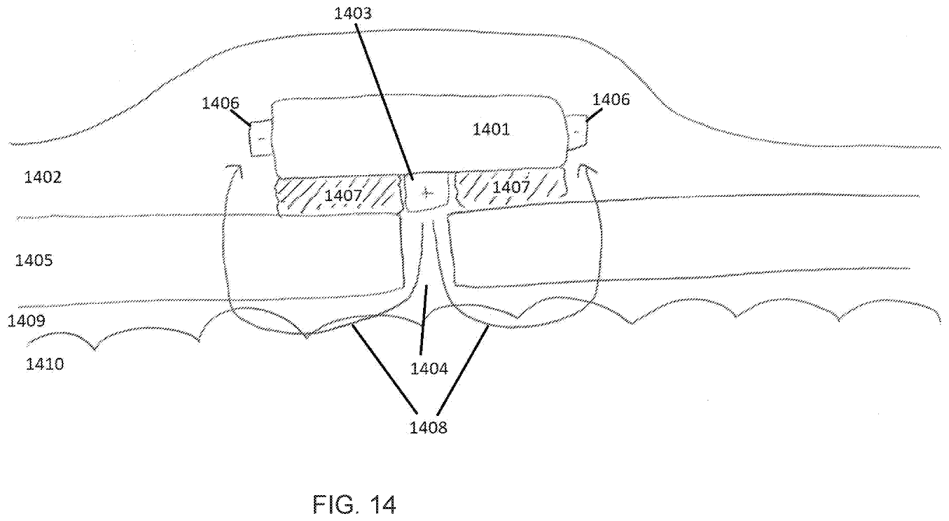

A drill hole may also be placed underneath the cathode. This allows electric current density to be more concentrated directly beneath the device, as well as lowering the overall impedance and resulting in a greater percentage of current flow reaching the sub-cranial space and affecting the brain. An example cross section of a device is shown in FIG. 14. In this example, a disk-shaped current generator (1401) is implanted beneath the scalp (1402). The device comprises a cathode (1403) which is positioned over a drill hole (1404) in the skull (1405). A ring-shaped anode (1406) is located on the outer edge of the current generator. An insulator (1407) having an impedance higher than the impedance of the bone in the skull is positioned around the cathode to prevent electric current from shunting from the cathode to the anode without entering the skull. When current is generated, a significant portion will follow the current path (1408) which flows from the cathode, through the drill hole into the sub-cranial space (1409) and into the brain (1410), then through the skull into the scalp to the anode.

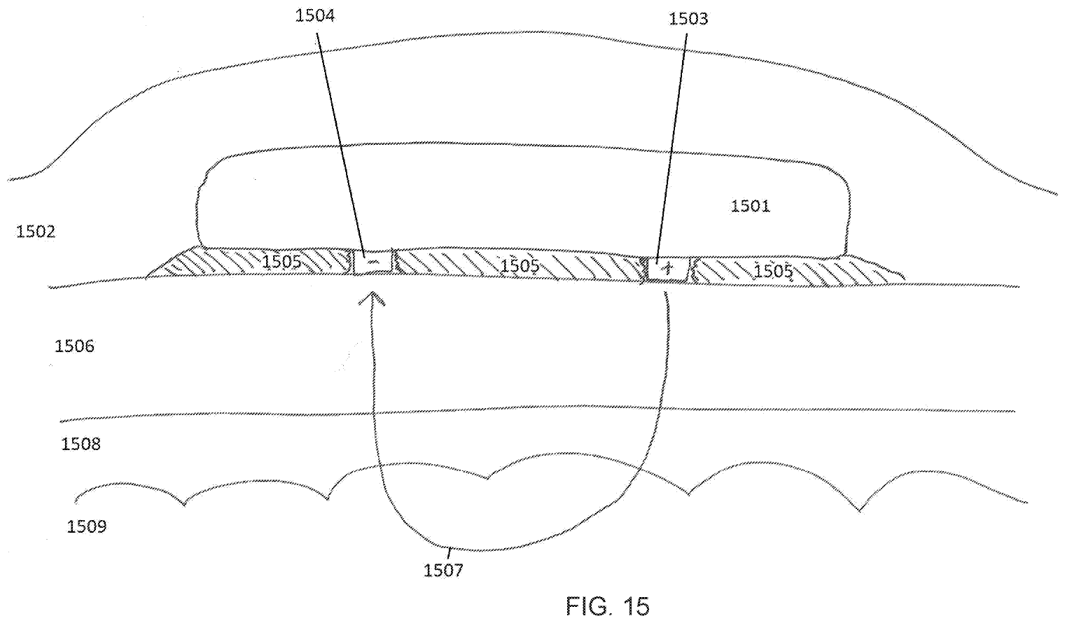

One issue that arises with an uninsulated subcutaneous electrode, as shown in FIG. 5 and FIG. 6, is that the current path is not well controlled, since the skull thickness is variable. For example, if the skull is very thin in one area, then the current density will tend to concentrate in that area going through the skull. One way to minimize the impact of skull thickness variability is to insulate both electrodes from the scalp, as shown in FIG. 1. FIG. 15 shows an example device in which both electrodes are insulated from each other and from the scalp tissue, exposed only on the side that is near the skull. In this example, the current generator (1501) is implanted under the scalp (1502). The cathode (1503) and anode (1504) are attached to the current generator. The device comprises an insulator (1505), which encircles both electrodes, and insulates them from each other and from the scalp tissues. In this configuration as well, the current generator is configured to prevent current from flowing from the cathode to the anode through the current generator, and the insulator has an impedance higher than the impedance of the bone in the skull. Thus, nearly all current flowing from the cathode to the anode must travel through the skull (1506). When current is generated, a significant amount will follow the current path (1507) from the cathode, through the skull into the sub-cranial space (1508) and through the brain (1509), then back through the skull to the anode. By extending the insulator beyond the current generator, the amount of current flowing through the scalp will be minimized. Also, by increasing the distance between the two electrodes, less current will shunt straight through the skull without first entering the sub-cranial space.

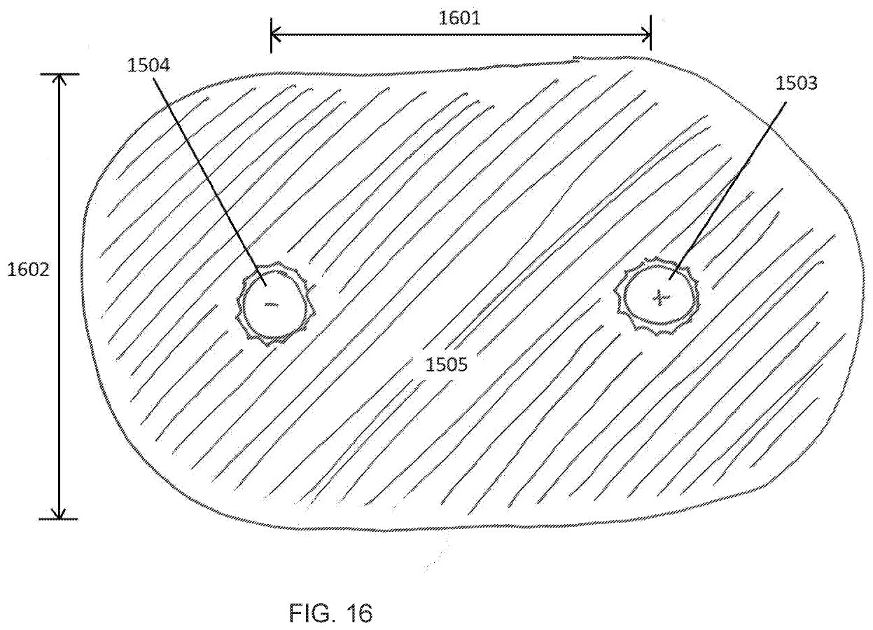

FIG. 16 shows the bottom side of the embodiment of FIG. 15. As shown, the cathode (1503) and the anode (1504) are attached to the bottom of the device, and the insulator (1505) surrounds both electrodes and covers the bottom of the device. If a space exists around the electrode that is not insulated, it may be advantageous to insert a conductive gel or paste or other conductive material into the space. This may help to lower the impedance of the path between the electrode and the skull. Also, if the insulator is malleable, it is less likely of spreading underneath the electrodes. If an increased distance between electrodes is desired, the device may be oval shaped, similar to a rectangle with semicircles on each end. In order to increase the distance between electrodes, the length of the rectangle (1601) is increased. In order to increase the distance between an electrode and the edge of the insulation, the width of the rectangle (1602), and thereby the diameter of the semicircle, is increased.

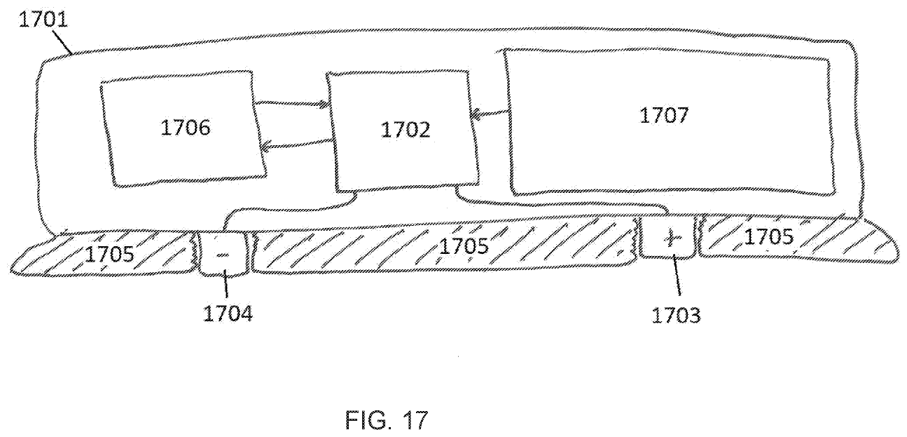

A cross section of an example device is shown in FIG. 17. In this, the current generator (1701) comprises a current source (1702), which generates electric current between the cathode (1703) and the anode (1704), which are both attached to the base of the current generator, with an insulator (1705) filling the space under the current generator and around the electrodes. The current source could be configured to be constant-current or constant-voltage. The current generator also comprises control logic (1706), which specifies parameters of stimulation. Stimulation may be pulsed or continuous current. The amplitude of the current could be fixed to a predetermined value or it may be variable. For example, the stimulation may be sinusoidal. If the stimulation amplitude is sinusoidal, then the frequency could be set to a prespecified value, such as an intrinsic frequency of an EEG band, such as the alpha frequency. The alpha frequency could be calculated from the user's EEG, or it may be a predefined desired alpha frequency, such as the alpha frequency of a generally healthy population or the average alpha frequency of a group of users. If stimulation is pulsed, the pulse frequency could be constant or it could be variable. The current generator in this example also comprises a power source (1707) in order to provide power to the current source and the control logic. This power source could be a battery or it could be a receiver for wireless power transfer with a power generator located external to the current generator. If the power source uses wireless power transfer, the power source could comprise a coil, which provides power through induction from an external alternating magnetic field.

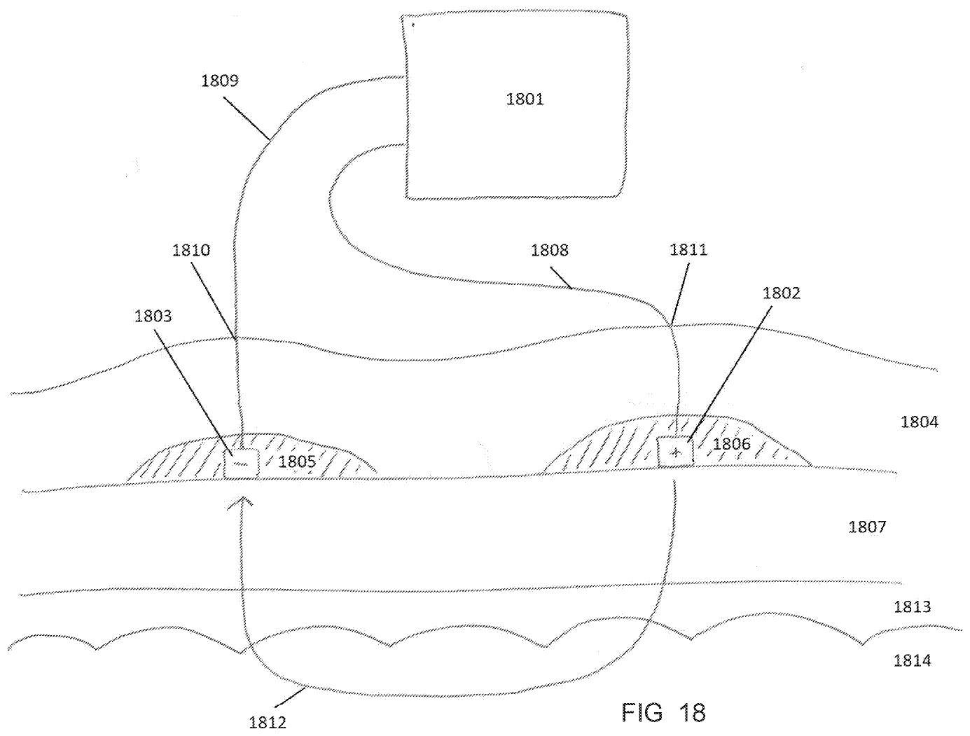

An alternative example is shown in FIG. 18. In this embodiment, the current generator (1801) is external to the body. The current generator may be a desktop unit, a cart-based unit, a handheld unit, or a unit which is wearable by the user, such as incorporated into a pendant or hat or backpack. The current generator is configured to generate a current between a cathode and an anode and to prevent current from flowing from the cathode to the anode through the current generator. In this example, a cathode (1802) and an anode (1803) are implanted beneath the subject's scalp (1804). The cathode and anode are each covered by an insulator (1805, 1806), which has an impedance greater than the impedance of the bone of the skull and therefore limits electric current flow from the cathode to the anode without passing through the skull (1807). The cathode and anode are each connected to the current generator by a wire (1808, 1809). The wires may be insulated and combined into a lead or they may be kept separate. The wires are percutaneous, and they pierce the scalp at locations (1810, 1811) near the electrodes (1802, 1803). When current is generated, a significant amount of current will follow the current path (1812) from the cathode, through the skull into the sub-cranial space (1813) and through the brain (1814), then back through the skull to the anode.

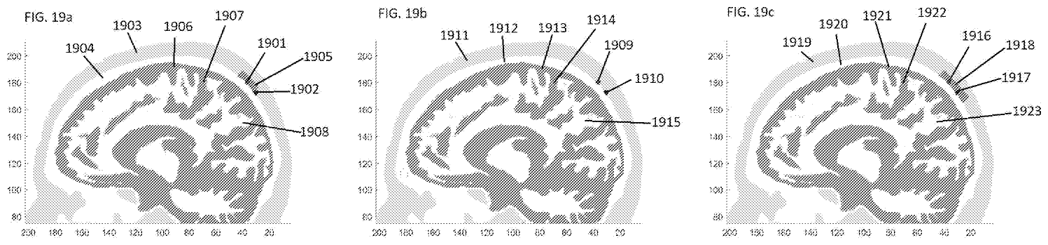

A simplified model of brain stimulation with an example of the present device is shown in FIGS. 19-21. Looking at FIG. 19a, this model comprises a cathode (1901) and an anode (1902), both implanted beneath the scalp (1903) and near the skull (1904), where an insulator (1905) is positioned around the cathode, such that nearly all electrical current generated from the cathode must proceed through the skull in order to flow to the anode. No insulation surrounds the anode. It is anticipated that a significant percentage of the current will flow through the sub-cranial space (1906) and through the brain gray matter (1907) and white matter (1908). In this model, the electrodes (1901, 1902) are approximately 2.5 mm wide and 1 mm thick and are disposed approximately 4 mm apart. The insulator is approximately 10.5 mm wide and 4 mm thick and has an impedance greater than the impedance of the bone of the skull. The skull is approximately 5 mm thick, and the scalp is approximately 8 mm thick. The electrodes are modeled as copper with conductivity 5.9e7 Siemens/meter (S/m). The white matter is modeled with conductivity 0.22 S/m. The gray matter is modeled with conductivity 0.47 S/m. The Cerebrospinal Fluid (CSF) is modeled with conductivity 1.71 S/m. The skull is modeled with conductivity 0.02 S/m. The scalp is modeled with conductivity 0.41 S/m. The insulator is modeled with conductivity of 1.0e-14 S/m, such that almost no electrical current will pass through it.

The model in FIG. 19a is compared with a model that is identical, except that the insulator is removed, shown in FIG. 19b. The figure shows the cathode (1909), the anode (1910), scalp (1911), skull (1912), sub-cranial space (1913), and the gray matter (1914) and white matter (1915) of the brain. In this model, current can easily shunt through the scalp from the cathode to the anode, without ever entering the skull.

The models in FIG. 19a and FIG. 19b are compared with a model that is identical, except that the insulator covers both electrodes, shown in FIG. 19c. The figure shows the cathode (1916), the anode (1917), the insulator (1918), scalp (1919), skull (1920), sub-cranial space (1921), and the gray matter (1922) and white matter (1923) of the brain. In this model, both the anode and cathode are insulated from the scalp and each other, requiring nearly all current to flow through the skull to reach either electrode.

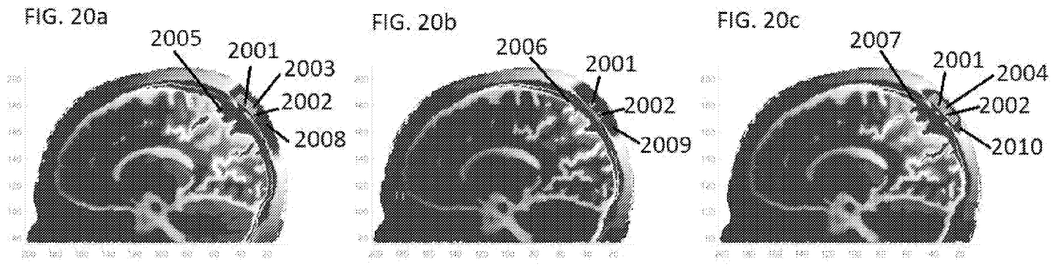

FIG. 20 shows a map of the current density for the three models shown in FIG. 19. In each of the figures, the dark area near the electrodes are indicated as areas of current density equal or above 0.16 A/m\hat{ }2. In FIG. 20, the cathode (2001) and anode (2002) are shown as small black squares. The insulator (2003) in FIG. 20a is shown as a larger gray rectangle surrounding the cathode (2001) on three sides. The insulator (2004) in FIG. 20c is shown as a larger gray rectangle surrounding the cathode (2001) and anode (2002) on three sides. In each image, the area of higher current density is shown under the skull (2005, 2007, 2009) and through the scalp (2006, 2008, 2010). It is clear that the current density under the skull does not extend deep into the brain in FIG. 20b, where the electrodes are not insulated. In this image, the majority of the high current density flows through the scalp. This configuration is least able to stimulate brain tissue. The current density in FIG. 20a and FIG. 20c both penetrate brain tissue a similar amount. However, the current through the scalp is much greater in FIG. 20a. This is reasonable, since the anode is insulated in the FIG. 20c embodiment and is not insulated in the FIG. 20a embodiment. Also, the current density is less symmetric in FIG. 20a. FIG. 20c shows deep penetration of brain tissue, much less current flow in the scalp, and more symmetric stimulation than either of the other two configurations.)

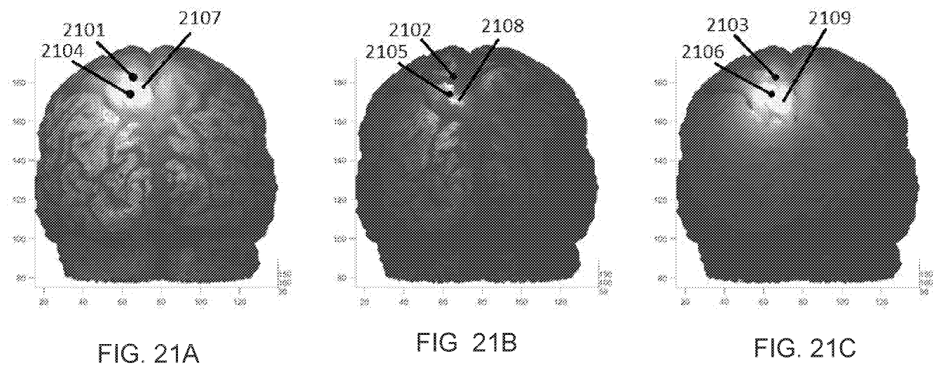

FIG. 21 shows a map of the current density for the three models, focusing only on the current penetrating the cortical surface. The three configurations in FIG. 21a-c correspond to the three configurations in FIG. 19a-c and FIG. 20a-c, respectively. The electrode locations for the cathode (2101, 2102, 2103) and the anode (2104, 2105, 2106) are shown as black circles. The light regions (2107, 2108, 2109) indicate the highest current density of at least 1.36 A/m\hat{ }2. From a visual inspection of the images, it is clear that the area of maximum current density (2108) is smallest in FIG. 21b, which is explained by the finding that the highest amount of scalp current flow occurs in the configuration without an insulator. The area of maximum current (2107) is largest in FIG. 21a. This is reasonable because the current will pass through the skull when returning to the anode (2104) in many locations, since current is unrestricted from flowing through the scalp when flowing to the anode. The area of maximum current (2109) is large, but focused for FIG. 21c. This is because current flow for both the cathode (2103) and anode (2106) are restricted from flowing through the scalp, and are forced to flow through the skull directly beneath the electrodes. The maximum intensity is highest in FIG. 21c.

In addition to stimulation, the device may incorporate EEG recording functionality, sensing signals using one electrode as the sense electrode and one as a reference. This system would further comprise a bio-amplifier and analog to digital converter. EEG recording is important in the diagnosis of mental disorders, to estimate functional damage (for example, due to a stroke or traumatic brain injury), or to map neuronal activity (for example, in seizure localization). In the treatment epilepsy, for example, a number of devices may be implanted and detect EEG activity showing a seizure to be imminent, and then provide stimulation to circumvent the seizure. EEG detection could be used to determine treatment parameter settings for the devices. For example, device stimulation may be applied only to the areas of the brain where the sensed EEG shows a possible functional deficit. In another example, the EEG recordings could be used to find an intrinsic frequency of an EEG band (for example, the alpha frequency), and then modulate the stimulation amplitude of one or more of the devices to stimulate at or near that frequency.

One way to sense EEG is to have both electrodes surrounded by an insulator, as in FIG. 19c. The impedance between the two electrodes is likely to be greatest in this configuration, effectively maximizing the input impedance of the EEG amplifier. This configuration has the additional advantage in that the measurement is made of the smallest region possible of the cortical surface, allowing for targeted EEG recording of a specific region of the brain.

The electrode material may be chosen to minimize the impedance of the interface between the electrode and the material that contacts it. In general, the electrode may contact scalp tissue, bone, CSF, blood, interstitial fluid, or some other body fluid. The material making contact with the body may be Titanium, Platinum, Platinum-Iridium, aluminum, stainless steel or some other biocompatible conductive material. A gel or paste may be used to reduce the impedance, to make the impedance more predictable, or to reduce infection or other complications from the implantation surgery. For example, a conductive gel may be used which comprises water, sodium chloride, Aragum, Potassium Bitartrate, Glycerin, Methylparaben, and Propylparaben. Gels containing these materials may reduce the impedance of the interface and may also soften and prepare surrounding tissues. In another example, an EEG paste may be used, which sticks to the electrode and reduces the impedance of the interface. The paste may be especially valuable for electrodes which are intended to touch or closely interface with the skull, since the paste may help hold the electrode while at the same time reducing the impedance. The insulator material may preferably be made of a material which provides long-term adhesive properties as well as having a high impedance to electric current flow. In one example, the insulator could be a bone void filler comprising hydroxyapatite and calcium sulfate. In another example, a silicone material may be used, which is preferably soft enough to conform to the surface variations of the skull. An adhesive insulator may be used which is heated before the surgical procedure, and hardens after implantation. If the device is disk-shaped, the insulator may be preinstalled on the device. Otherwise, it could be applied by the person performing the implantation. If the insulator also acts as an adhesive and the device is disk-shaped, then the insulator could hold the device in place as well as acting as an insulator. If the insulator does not have sufficient adhesive properties (silicone, for example), then the device may be held in place using a separate adhesive or with small screws. In a preferred embodiment, the device is disk-shaped, with an adhesive-insulating paste coating the bottom of the current generator, surrounding the central electrode. When the device is pressed into place during implant, the adhesive holds the device in place and provides insulation. In order to remove a device which is held in place using an adhesive or cement material, then a lever or other tool may be necessary to break the seal and remove the device.

The devices may communicate wirelessly in order to control stimulation. For example, the devices could incorporate Bluetooth communication technology, which would allow them to interact with another device that uses Bluetooth, such as a mobile phone running an application. The phone could also display EEG signals and show status information, such as battery life or electrode impedance.

When a feature or clement is herein referred to as being “on” another feature or clement, it can be directly on the other feature or element or intervening features and/or elements may also be present. In contrast, when a feature or element is referred to as being “directly on” another feature or element, there are no intervening features or elements present. It will also be understood that, when a feature or clement is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for electrical stimulation of a subject's brain, the device comprising:
a first electrode adapted to be implanted under a scalp of the subject and outside the subject's cranial cavity;
a second electrode adapted to be implanted under the subject's scalp and outside the subject's cranial cavity;
insulating material surrounding a portion of the first electrode, the insulating material having an impedance higher than an impedance of a human skull; and
a current generator adapted to generate an electric current between the first electrode and the second electrode.

2. The device of claim 1 further comprising insulating material surrounding a portion of the second electrode, the insulating material having an impedance higher than the impedance of a human skull.

3. The device of claim 1 wherein the first electrode and the insulating material are disposed on a first side of the current generator.

4. The device of claim 3 wherein the second electrode is disposed on the first side of the current generator.

5. The device of claim 3 wherein the second electrode is disposed on a second side of the current generator.

6. The device of claim 1 wherein the insulating material extends a uniform distance from a circumference of the first electrode.

7. The device of claim 1 wherein the insulating material extends in non-uniform distances from a circumference of the first electrode.

8. The device of claim 1 wherein the first electrode comprises a screw adapted to be screwed into the subject's skull.

9. A method for providing electrical stimulation to brain tissue of a subject, the method comprising:
generating a current flow path between a first electrode disposed below a scalp of the subject and outside a cranial cavity of the subject to a second electrode disposed below the scalp and outside the cranial cavity; and
directing the current flow path through the subject's skull and into the brain tissue.

10. The method of claim 9 wherein the directing step comprises resisting current flow between the first electrode and the second electrode outside of the skull with insulating material having an impedance higher than an impedance of the skull.

11. The method of claim 10 wherein the insulating material surrounds a portion of the first electrode.

12. The method of claim 11 wherein the insulating material surrounds a portion of the second electrode.

13. The method of claim 10 wherein the method further comprises placing the insulating material against an outside surface of the skull between the first electrode and the second electrode.

14. The method of claim 9 wherein the generating step comprises generating current with a current generator disposed between the scalp and the skull.

15. The method of claim 9 further comprising screwing the first electrode into the skull.

16. The method of claim 9 further comprising creating an opening through the skull to the cranial cavity adjacent the first electrode or the second electrode.

17. A method for obtaining an EEG of a subject's brain, the method comprising:
sensing an electroencephelography signal from a first electrode disposed below a scalp of the subject and outside a cranial cavity of the subject and a second electrode disposed below the scalp and outside the cranial cavity; and
recording the electroencephelography signal.

18. The method of claim 17 wherein at least a portion of the first electrode and/or the second electrode is surrounded by an insulating material.
